(19) United States
(12) Patent Application Publication
(10) Pub. No.: US 2026/0223966 A1
(43) Pub. Date: Aug. 6, 2026

(54) FALSE-EYELASH MAGNETIC WEARING SYSTEM AND APPLICATION THEREOF

(71) Applicant: Tuchuang Xinglian (Qingdao) Innovation Technology Co., Ltd., Shandong (CN)

(72) Inventor: Feng GENG, Shandong (CN)

(73) Assignee: Tuchuang Xinglian (Qingdao) Innovation Technology Co., Ltd., Shandong (CN)

(21) Appl. No.: 19/651,745

(22) Filed: Apr. 19, 2026

(51) Int. Cl. A41G 5/02; A61K 8/19; A61K 8/36; A61K 8/81; A61Q 1/10

(52) U.S. Cl. CPC A41G 5/02; A61K 8/19; A61K 8/361; A61K 8/8147; A61Q 1/10; A61K 2800/43; A61K 2800/47

(57) ABSTRACT

Disclosed are a false-eyelash magnetic wearing system and an application thereof, the system comprising: (a) an eyelash magnetization fluid, the preparation raw materials of which comprise: an acrylates copolymer, a magnetic pigment, and water, a mass content of the acrylates copolymer in the raw materials being 10%-30%, and a mass content of the magnetic pigment in the raw materials being 50%-80%, with the balance being water; (b) magnetic block-equipped false eyelashes, comprising: a non-magnetic flexible base strip; false eyelash fibers fixed to the flexible base strip; and a plurality of permanent magnet blocks fixed to the flexible base strip, the plurality of permanent magnet blocks being arranged along the length direction of the flexible base strip, and the permanent magnet blocks being used to generate a magnetic attraction force with natural eyelashes treated by the eyelash magnetization fluid.

### TECHNICAL FIELD

The present disclosure relates to the technical field of beauty makeup tools, and more specifically, to a false-eyelash magnetic wearing system and an application thereof.

### BACKGROUND

Magnetic false eyelashes are increasingly favored by consumers due to their convenience. Currently, magnetic false eyelashes on the market are mainly divided into two categories:

The first category is the “sandwich type,” which requires two sets of false eyelashes, both equipped with magnets, to be magnetically attracted to each other, sandwiching the user's natural eyelashes in between. However, this method results in heavy false eyelashes overall, creating significant pressure on the eyelids and natural eyelashes, and the wearing process is complex and uncomfortable.

The second category is the “magnetic eyeliner adsorption type.” For example, US patent No. US20210093031A1 discloses a solution that requires applying a special eyeliner containing magnetic particles onto the eyelid skin, and then attaching a false eyelash strip that is entirely magnetic or magnetically attractable to the eyeliner. The disadvantages of this solution are: the user must draw eyeliner, limiting makeup effects; adsorption depends on skin condition and is prone to falling off due to oil or sweat; and to possess overall magnetism, the false eyelash strip is usually quite rigid, causing a strong sensation of a foreign object when worn.

Furthermore, in recent years, the industry has begun to explore introducing magnetic particles directly into mascara formulas to magnetize the natural eyelashes themselves. For example, US patent publication No. US20200281828A1 discloses a load-bearing magnetic mascara composition and a magnetic false-eyelash system used in conjunction with it. Specifically, the patent discloses an eyelash magnetization fluid containing 10%-60% magnetic particles (such as black iron oxide) and structural adhesives such as acrylic copolymers, which forms a magnetic film after being applied to natural eyelashes; it is used in conjunction with false eyelashes that have magnetic elements combined at the base of the eyelashes, achieving attachment through magnetic force. However, its technical solution still has the following technical problems:

First, wearing comfort still needs improvement. Although the false eyelashes in this patent uses magnetic elements, the description in its drawings and specification regarding the arrangement of magnetic elements at the eyelash base is relatively broad and does not specifically optimize the distribution form of the magnets. In practice, if continuous magnetic strips or large magnetic blocks are used, the overall flexibility of the false eyelashes is limited, and the sensation of a foreign object on the eyelid remains significant.

Second, the magnetic response efficiency and film-forming stability of the magnetic particles need to be improved. This patent uses a single iron oxide black (Fe₃O₄) as the magnetic pigment. When added at a high content, it is prone to particle agglomeration and uneven dispersion, which affects the uniformity and magnetic response intensity of the magnetic film after drying, thereby affecting the adsorption firmness of the false eyelash.

Third, there is still room for optimization in the selection and ratio of formula components. The magnetic particle content in this patent is 10%-60%, which is a broad range, but it does not conduct in-depth research and optimization on the synergistic effect of specific humectants, surfactants, and high-content magnetic particles, which may lead to deficiencies in the product's long-term storage stability and skin-feel experience during use.

### SUMMARY

In view of the deficiencies of the prior art mentioned above, the present disclosure provides a false-eyelash magnetic wearing system and application that is more natural, comfortable, and firm to wear, and does not rely on eyelid skin. By optimizing the structure of the false eyelashes (adopting discrete magnetic blocks+flexible base strip) and simultaneously improving the formula of the eyelash magnetization fluid (e.g., introducing a composite magnetic material with a specific ratio, and optimizing the combination of humectants and surfactants), the present disclosure aims to solve the technical problems existing in the prior art, such as poor comfort, insufficient magnetic response efficiency, and the need for improved formula stability.

To achieve the above objectives, the present disclosure adopts the following technical solutions:

In a first aspect, a false-eyelash magnetic wearing system includes:
(a) an eyelash magnetization fluid, the eyelash magnetization fluid being prepared from the following raw materials: an acrylates copolymer, a magnetic pigment, and water, wherein a mass content of the acrylates copolymer in the raw materials is 10%-30%, a mass content of the magnetic pigment in the raw materials is 50%-80%, with the balance being water;
(b) magnetic block-equipped false eyelashes, including:
a non-magnetic flexible base strip;
false eyelash fibers fixed to the flexible base strip; and
a plurality of permanent magnet blocks fixed to the flexible base strip, the plurality of permanent magnet blocks being arranged along a length direction of the flexible base strip, and the permanent magnet blocks being configured to generate a magnetic attraction force with natural eyelashes treated by the eyelash magnetization fluid.

Preferably, in the eyelash magnetization fluid, the mass content of the acrylates copolymer in the raw materials is 20%-30%; and/or, the mass content of the magnetic pigment in the raw materials is 65%-75%.

Optionally, the acrylates copolymer includes but is not limited to: EMUL 33, EMUL EF-1, AquaStyle™ SH-100, Film Former, acrylates/stearyl acrylate/dimethicone methacrylate copolymer (KP-561P), acrylic acid/methyl methacrylate/butyl acrylate copolymer, acrylic acid/methacrylic acid/ethyl acrylate copolymer, and acrylic acid/methacrylic acid/acrylate copolymer.

Preferably, the magnetic pigment is selected from a group consisting of: black iron oxide, fatty acid surface-treated black iron oxide, nano-scale ferroferric oxide, composite magnetic material, or a mixture of a hard magnetic material and a soft magnetic material; wherein the composite magnetic material is a Fe₃O₄@SiO₂ core-shell structure material; wherein the hard magnetic material is at least one of barium ferrite or strontium ferrite; and the soft magnetic material is cobalt-nickel ferrite.

Preferably, the nano-scale ferroferric oxide has a particle size from 20 to 100 nm; or the fatty acid is selected from at least one of lauric acid or oleic acid; or in the mixture of a hard magnetic material and a soft magnetic material, a mass percentage of the hard magnetic material to the soft magnetic material is 60%-80%: 20%-40%.

Preferably, the raw materials of the eyelash magnetization fluid further include a humectant, a surfactant, and a pH regulator; the humectant is selected from at least one of propylene glycol, butylene glycol, glycerin, dipropylene glycol, PEG400, sorbitol, sodium hyaluronate, sodium PCA, trehalose, or methyl gluceth-20, with a mass content of 2%-6% in the raw materials; the surfactant is selected from at least one of polysorbate-80, poloxamer 188, poloxamer 407, lecithin, diethylhexyl phosphate, dibutyl phosphate, or lauric acid, with a mass content of 1%-3% in the raw materials; and the pH regulator is selected from arginine, with a mass content of 0.5%-2% in the raw materials.

Further preferably, the humectant is selected from: a combination of butylene glycol with a mass content of 1%-2% and glycerin with a mass content of 1%-2% in the raw materials, or a combination of 1%-2% methyl gluceth-20, 0.1%-0.2% sodium hyaluronate, and 2%-3% butylene glycol; and/or,
the surfactant is selected from a combination of poloxamer 188 with a mass content of 1%-2% and lecithin with a mass content of 0.5%-1% in the raw materials.

Preferably, the false-eyelash magnetic wearing system further includes a preservative, wherein the preservative is selected from at least one of ethylhexylglycerin and caprylyl glycol, with a mass content of 0.1%-1% in the raw materials.

Preferably, the raw materials of the eyelash magnetization fluid include, by mass percentage: 22% to 25% of an acrylates copolymer, 65% to 68% of a magnetic pigment, 2%-6% of a humectant, 2%-3% of a surfactant, 0.8%-1.5% of a pH regulator, 0.1%-0.5% of a preservative, with the balance being water.

Optionally, a method for preparing the eyelash magnetization fluid includes the following steps:
Step 1: mixing the acrylates copolymer with water, then adding the magnetic pigment and mixing;
Step 2: mixing a mixture from step 1 at a speed of 800-8000 rpm for 5-15 min;
Step 3: further adding the humectant, the surfactant, and the pH regulator to the mixture and mixing, and finally adding the preservative and mixing;
Step 4: allowing the mixture to stand, and filtering out solids to obtain the eyelash magnetization fluid.

Preferably, a vacuum defoamation treatment is added after step 2 or step 3.

Preferably, in step 2, the mixture from step 1 is mixed using a high-speed shear emulsification process at a speed of 3000-6000 rpm for 5-8 min; and/or,
in step 4, a 200-400 mesh screen is used for filtration to remove large particle agglomerates and impurities.

Preferably, a material of the non-magnetic flexible base strip is silicone, thermoplastic elastomer, or fabric; preferably, the thermoplastic elastomer is selected from at least one of thermoplastic polyurethane elastomer, thermoplastic polyolefin elastomer, thermoplastic polyester elastomer, and thermoplastic vulcanizate; and wherein the fabric is selected from one of natural fiber fabric, synthetic fiber fabric, non-woven fabric, and blended fabric.

Optionally, the natural fiber fabric includes cotton cloth, silk, etc., which has good skin affinity but requires moisture-proof and anti-mildew treatment. Synthetic fiber fabrics include polyester fiber (dacron): high strength, wear-resistant, and not easily deformed; polyamide (nylon): soft, good elasticity, and fatigue-resistant; spandex (elastic fiber): high elasticity, often blended with polyester or polyamide to improve the fit of the base strip. Blended fabrics such as cotton-polyester blends, nylon-spandex blends, etc., balance comfort, strength, and elasticity. Non-woven fabrics are made of polypropylene (PP), polyester (PET), etc., which are lightweight and breathable, suitable for disposable or short-term use false-eyelash base strips.

Preferably, the permanent magnet blocks are arranged at intervals along a length direction of the flexible base strip; the permanent magnet blocks are wrapped or bonded within the flexible base strip, or fixed to a surface of the flexible base strip; and a number of the permanent magnet blocks is 2 to 5.

Further preferably, an anti-corrosion coating, preferably a nickel-plated or gold-plated layer, is provided on a surface of the permanent magnet block; or the permanent magnet block is coated with a medical-grade silicone layer.

Further preferably, the permanent magnet block has a magnetic micro-array structure, including a plurality of magnetic microcolumns or microgrooves; and magnetic pole directions of the plurality of permanent magnet blocks are arranged alternately.

In a second aspect, the present disclosure provides a method for wearing false eyelashes using the false-eyelash magnetic wearing system according to the first aspect, including the following steps:
applying the eyelash magnetization fluid onto a user's natural eyelashes;
bringing the permanent magnet blocks of the magnetic block-equipped false eyelash close to the natural eyelashes coated with the eyelash magnetization fluid, and adsorbing and fixing the magnetic block-equipped false eyelashes onto the natural eyelashes through magnetic force.

Preferably, the method further includes a step of adsorbing and fixing the magnetic block-equipped false eyelashes onto an upper or lower side of the natural eyelashes according to makeup requirements.

Preferably, the method further includes segmented wearing: selecting false eyelashes with different numbers of magnetic blocks for separate wearing according to different regions of an inner corner, a middle portion, and an outer corner of an eye; or combined wearing: wearing the magnetic block-equipped false eyelashes onto both the upper and lower sides of the natural eyelashes simultaneously.

Further preferably, the method further includes a makeup removal step: using an eye and lip makeup remover to wipe off the eyelash magnetization fluid on the natural eyelashes, or using a magnetic makeup removal wand to assist in removing the false eyelashes.

In a third aspect, the present disclosure provides a method for preparing the eyelash magnetization fluid used in the false-eyelash magnetic wearing system according to the first aspect, including:
step (1), mixing the acrylates copolymer with water, and then adding the magnetic pigment and mixing;
step (2), mixing a mixture obtained in step (1) at a rotation speed of 800-8000 rpm for 5-15 min;
Step (3), afterwards leaving a mixture obtained in step (2) to stand, and obtaining the eyelash magnetization fluid after filtering out solids.

Preferably, the eyelash magnetization fluid further includes a humectant, a surfactant, a pH regulator, and a preservative; and the preparation method further includes: after the step (2), adding and mixing the humectant, the surfactant, and the pH regulator, then adding and mixing the preservative; and then performing the step (3).

Further preferably, a vacuum defoamation treatment is added after the step (2) or the step (3).

Compared with the prior art, the present disclosure has the following beneficial effects:

Firstly, the present disclosure creatively proposes a technical path of “magnetizing natural eyelashes” rather than “magnetizing the skin or the entire false eyelashes.” It solves the problems of the “eyeliner adsorption type” (US20210093031A1) which relies on drawing eyeliner and skin condition, and is uncomfortable to wear. At the same time, the present disclosure adopts a structure of “independent magnetic blocks+ordinary false eyelashes,” which also avoids the heaviness and uncomfortable experience of the “sandwich type” that requires two pairs of magnetic false eyelashes.

Second, wearing false eyelashes using the method of the present disclosure is extremely natural, comfortable, and burden-free. The eyelash magnetization fluid of the present disclosure, through the design of a flexible base strip and discrete small magnetic blocks, makes the false eyelashes lightweight and soft as a whole, avoiding the pressure on the eyelids caused by rigid magnetic strips or double-layered false eyelashes, resulting in minimal foreign body sensation and comfort even during prolonged wear. Furthermore, since there is no need to apply any colored substances to the skin, and the false eyelashes are magnetically attached to one's own natural eyelashes, a natural integration of false eyelashes and natural eyelashes is achieved, making the makeup invisible and capable of achieving a “no-makeup” look, meeting makeup needs for different scenarios.

Third, by applying the eyelash magnetization fluid of the present disclosure to eyelashes, the magnetic force acts directly on the magnetic particles on the natural eyelashes, providing a direct and strong adsorption force. Furthermore, as it does not rely on the skin, it is unaffected by factors such as eyelid oil, sweat, or tears, significantly improving stability and providing a more secure wearing experience.

Fourth, the wearing method of the present disclosure is flexible and diverse: users can freely choose to attach the false eyelashes above the natural eyelashes (to increase density) or below them (to provide support and a curling effect) based on their own eyelash conditions, eye shapes, and makeup needs; they can even perform segmented or upper-lower combined wearing to create personalized makeup effects.

### BRIEF DESCRIPTION OF DRAWINGS

The drawings, which constitute a part of this application, are provided for a further understanding of the present disclosure. The illustrative embodiments and the descriptions thereof are used to explain the present disclosure and do not constitute an improper limitation thereof. In the drawings:

FIG. 1 is a schematic structural diagram of magnetic block-equipped false eyelashes of the false-eyelash magnetic wearing system of the present disclosure.

FIG. 2 is another schematic structural diagram of the magnetic block-equipped false eyelashes of the present disclosure.

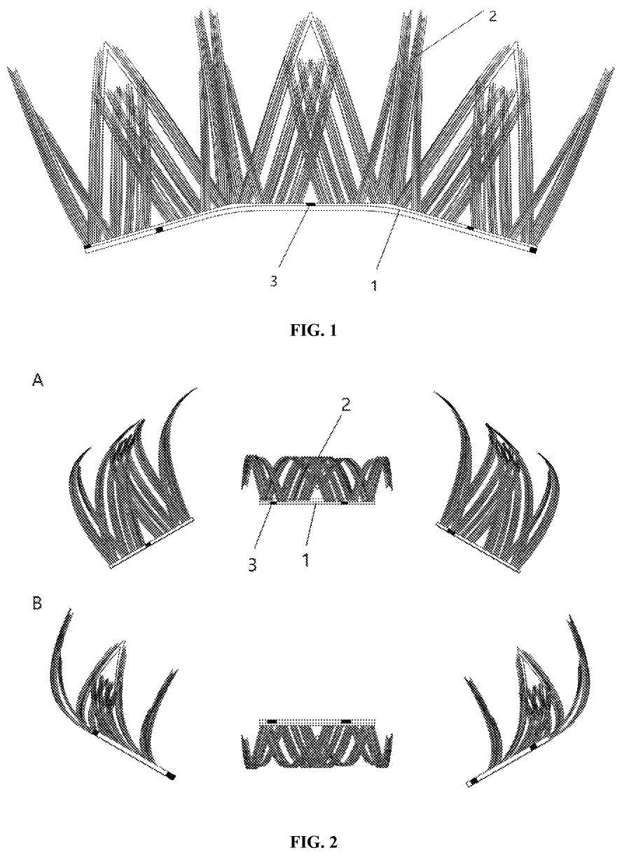

In FIG. 1 and FIG. 2, 1: Flexible base strip, 2: False eyelash fiber, 3: Magnetic block.

### DESCRIPTION OF EMBODIMENTS

The acrylates copolymers that may be used in the present disclosure include, but are not limited to: EMUL 33, EMUL EF-1, AquaStyle™ SH-100, Film Former, acrylates/stearyl acrylate/dimethicone methacrylate copolymer (KP-561P), acrylic acid/methyl methacrylate/butyl acrylate copolymer, acrylic acid/methacrylic acid/ethyl acrylate copolymer, and acrylic acid/methacrylic acid/acrylate copolymer, all of which are commercially available brands. EMUL 33 and EMUL EF-1 are mainly used as rheology modifiers to provide thickening, suspending, and co-emulsifying functions; AquaStyle™ SH-100 is a multi-purpose shape-retaining and styling polymer, mainly used as a film-forming agent; Film Former is an emulsion polymer with 30% active content, low VOC, and good film-forming properties; KP-561P is an organosilane-modified acrylic polymer wax with a melting point of 25-35° C., which can improve skin feel, gloss, and other properties; acrylic acid/methyl methacrylate/butyl acrylate copolymer, acrylic acid/methacrylic acid/ethyl acrylate copolymer, and acrylic acid/methacrylic acid/acrylate copolymer are mainly used as thickeners and film-forming agents to enhance water resistance, skin feel, and other properties.

Among the optional components of the above-mentioned acrylates copolymers, it is preferred to use thickening and suspending types (such as EMUL 33, EMUL EF-1): which help stabilize the dispersion system of high-content magnetic pigment and prevent sedimentation; or film-forming and setting types (such as AquaStyle™ SH-100): which provide good film-forming properties and flexibility, ensuring that the magnetic film layer is firm and not easy to fall off.

The technical solutions in the embodiments of the present disclosure will be clearly and completely described below in conjunction with the preferred schemes of the present disclosure and the related drawings. Obviously, the described embodiments are a part of the embodiments of the present disclosure, rather than all of the embodiments. The following description of at least one exemplary embodiment is actually illustrative and is by no means intended to limit the present disclosure and an application thereof or use. Based on the embodiments in the present disclosure, all other embodiments obtained by those of ordinary skill in the art without creative labor shall fall within the protection scope of the present disclosure.

It should be noted that the terminology used herein is for the purpose of describing specific embodiments only and is not intended to limit the exemplary embodiments according to the present application. As used herein, the singular forms are intended to include the plural forms as well, unless the context clearly indicates otherwise. Furthermore, it should be understood that when the terms “include” and/or “include” are used in this specification, they specify the presence of features, steps, operations, devices, components, and/or combinations thereof.

Unless specifically stated otherwise, the relative arrangements, numerical expressions, and values of the components and steps set forth in these embodiments do not limit the scope of the present disclosure. Meanwhile, it should be understood that for the convenience of description, the dimensions of the various parts shown in the drawings are not drawn to actual scale. Techniques, methods, and equipment known to those of ordinary skill in the relevant art may not be discussed in detail, but where appropriate, such techniques, methods, and equipment should be considered as part of the authorized specification. In all examples shown and discussed herein, any specific values should be interpreted as exemplary rather than as limiting. Therefore, other examples of the exemplary embodiments may have different values. It should be noted that similar reference numerals and letters represent similar items in the following drawings; therefore, once an item is defined in one drawing, it does not need to be further discussed in subsequent drawings.

### Eyelash Magnetization Fluid Formula

### Example 1-1

This embodiment provides an eyelash magnetization fluid, the preparation raw materials of which consist of: EMUL EF-1: 30 g, iron oxide black (Fe₃O₄, with an average particle size of 5 μm): 65 g, water: 5 g.

Preparation method: EMUL EF-1 was mixed with water, iron oxide black was added, followed by stirring at 1500 rpm for 10 min; the obtained mixture was left to stand to defoam, and then filtered through a 200-400 mesh screen to remove large particle aggregates and impurities to obtain the eyelash magnetization fluid.

### Example 1-2

This embodiment provides an eyelash magnetization fluid. Based on a total raw material weight of 100 g, its composition is: EMUL EF-1: 23 g, black iron oxide (Fe₃O₄, D50=3 μm): 68 g, butanediol: 2 g, glycerin: 1.5 g, poloxamer 188:1.5 g, lecithin: 0.5 g, arginine: 1 g, and water as the balance.

Preparation method: EMUL EF-1 was mixed with water, iron oxide black was added, followed by stirring at 1000 rpm for 8 min; butylene glycol, glycerin, poloxamer 188, lecithin, and arginine were added, and stirring was continued for 5 min; the obtained mixture was left to stand to defoam, and then filtered through a 200-400 mesh screen to remove large particle aggregates and impurities to obtain the eyelash magnetization fluid.

### Example 1-3

This embodiment provides an eyelash magnetization fluid. Based on a total raw material weight of 100 g, its composition is: AquaStyle™ SH-100: 22 g, a magnetic pigment (Fe₃O₄@SiO₂ core-shell structure, a commercially available product, with a core diameter of 50 nm, a shell thickness of 5 nm, D50-2 μm): 65 g, methyl gluceth-20: 2 g, sodium hyaluronate: 0.15 g, butylene glycol: 2.5 g, poloxamer 188:1.5 g, lecithin: 0.8 g, arginine: 1.2 g, ethylhexylglycerin: 0.3 g, caprylyl glycol: 0.2 g, and water as the balance.

Preparation method: the acrylates copolymer was mixed with water, the magnetic pigment was added, and the obtained mixture was dispersed at 4000 rpm for 8 minutes using a high-speed shear emulsification process, followed by vacuum defoamation treatment for 10 minutes; methyl gluceth-20, sodium hyaluronate, butylene glycol, poloxamer 188, lecithin, and arginine were further added, and high-speed shear stirring was continued for 5 minutes; ethylhexylglycerin and caprylyl glycol were added, and high-speed shear stirring was continued for 3 minutes; then vacuum defoamation treatment was performed for 10 minutes, and the mixture was filtered through a 200-400 mesh screen to remove large particle aggregates and impurities to obtain the eyelash magnetization fluid.

### Example 1-4

This embodiment provides an eyelash magnetization fluid. Based on a total raw material weight of 100 g, its composition is: an acrylates copolymer: 24 g, a magnetic pigment (barium ferrite: cobalt nickel ferrite=70%: 30%): 66 g, butylene glycol: 2 g, glycerin: 2 g, poloxamer 188: 1 g, lecithin: 0.5 g, arginine: 1%, ethylhexylglycerin: 0.3%, caprylyl glycol: 0.2%, and water as the balance.

Preparation method: the acrylates copolymer was mixed with water, the magnetic pigment was added, and the obtained mixture was dispersed at 6000 rpm for 5 minutes using a high-speed shear emulsification process, followed by vacuum defoamation treatment for 10 minutes; methyl gluceth-20, sodium hyaluronate, butylene glycol, poloxamer 188, lecithin, and arginine were further added, and high-speed shear stirring was continued for 5 minutes; ethylhexylglycerin and caprylyl glycol were added, and high-speed shear stirring was continued for 3 minutes; then vacuum defoamation treatment was performed for 10 minutes, and the mixture was filtered through a 200-400 mesh screen to remove large particle aggregates and impurities to obtain the eyelash magnetization fluid.

### Magnetic Block-Equipped False Eyelashes

### Example 2-1

This example provides magnetic block-equipped false eyelashes, including:
a non-magnetic flexible base strip: made of medical-grade liquid silicone, injection-molded into a soft strip with a semi-circular cross-section, a length of 2.5 cm, a width of 1.2 mm, and a thickness of 0.8 mm;
false eyelash fibers: made of PBT synthetic fiber, approximately 15 fibers per bundle, bonded at equal intervals to the upper surface of the silicone base strip using medical-grade glue, totaling 8 bundles;
permanent magnet blocks: made of a NdFeB material, with dimensions of 2 mm×1 mm×1 mm, having a nickel-plated surface for corrosion resistance. Three magnetic blocks were arranged at equal intervals along a length direction of the base strip, embedded inside the silicone base strip through injection molding, with the side of the magnetic block facing the natural eyelash exposed.

### Example 2-2

This example provides magnetic block-equipped false eyelashes, including:
a non-magnetic flexible base strip: made of thermoplastic polyurethane elastomer (TPU), extrusion-molded into a soft strip with a circular cross-section, 3 cm in length and 0.9 mm in diameter;
false eyelash fibers: made of natural hair, fixed to the upper surface of the TPU base strip through hot-melt bonding, totaling 10 bundles;
permanent magnet blocks: made of a NdFeB material, with a gold-plated surface for corrosion resistance, coated with a medical-grade silicone layer. The magnetic blocks featured a magnetic micro-array structure, with multiple microcolumns (a height of 0.3 mm, a diameter of 0.2 mm) on the surface of each magnetic block to enhance contact uniformity with the magnetic film. There were 4 magnetic blocks in total, fixed inside the base strip through hot-pressing, with magnetic poles arranged in an alternating pattern (N-S-N-S) to enhance magnetic flux penetration.

### Example 2-3

This example provides magnetic block-equipped false eyelashes, including:
a non-magnetic flexible base strip: made of polyamide-spandex blended fabric (85% polyamide+15% spandex), with a width of 1.5 mm, where the length could be trimmed according to eye shape, featuring high elasticity and fit;
False eyelash fibers: made of polyester fiber, fixed to the upper surface of the fabric base strip through sewing;
permanent magnet blocks: made of a NdFeB material, with dimensions of 1.5 mm×1.5 mm×1 mm, and a nickel-plated surface. There were 5 magnetic blocks in total, bonded to the side of the base band facing the natural eyelash using medical-grade soft glue, facilitating easy removal and replacement.

### False-Eyelash Magnetic Wearing System

### Example 3-1

This example provides a method for wearing false eyelashes using the false-eyelash magnetic wearing system, including:
Step 1: using a brush head to evenly apply the eyelash magnetization fluid prepared in Example 1-3 onto the natural eyelashes of the upper eyelid, brushing one layer from the root to the tip, and waiting about 45 seconds for it to dry;
Step 2: using tweezers to pick up the magnetic block-equipped false eyelashes prepared in Example 2-2, and gently bringing the side with the permanent magnet blocks close to and placing it over the treated natural eyelashes;
Step 3: after automatic magnetic attraction, using small tweezers to gently adjust the curvature and position of the false eyelashes to fit the eye shape.

### Example 3-2

This example provides a method for wearing false eyelashes using the false-eyelash magnetic wearing system, including:
Step 1: using a brush head to evenly apply the eyelash magnetization fluid prepared in Example 1-4 onto the natural eyelashes of the upper eyelid, brushing 2 layers with a 30-second interval between each layer, and waiting about 60 seconds for it to dry;
Step 2: using tweezers to pick up the magnetic block-equipped false eyelashes prepared in Example 2-1, and gently supporting the side with the permanent magnet blocks underneath the treated natural eyelashes;
Step 3: after magnetic attraction, adjusting the position to achieve support and curling effects.

### Example 3-3

This example provides a method for wearing false eyelashes using the false-eyelash magnetic wearing system, including:
Step 1: evenly applying the eyelash magnetization fluid prepared in Example 1-2 onto the natural eyelashes of the upper eyelid, and allowing it to dry;
Step 2: according to eye shape requirements, selecting false eyelashes with different numbers of magnetic blocks:
Inner corner area: using short false eyelashes with 1 magnetic block;
Middle eye area: using standard false eyelashes with 3 magnetic blocks;
Outer corner area: using long false eyelashes with 2 magnetic blocks;
Step 3: adsorbing and fixing each segment of the false eyelashes to the corresponding area to achieve a personalized makeup effect.

### Example 3-4

This example provides a method for wearing false eyelashes using the false-eyelash magnetic wearing system, including:
Step 1: applying the eyelash magnetization fluid prepared in Example 1-3 evenly onto the natural eyelashes of the upper eyelid, and allowing it to dry;
Step 2: taking two pairs of magnetic block-equipped false eyelashes prepared in Example 2-2 respectively;
Step 3: adsorbing and fixing one pair onto the upper side of the natural eyelashes and the other pair onto the lower side, forming a sandwich-style adsorption effect to enhance density and curl.

FIG. 1 and FIG. 2 respectively show structural schematic diagrams of the magnetic block-equipped false eyelash. Depending on the wearing methods in Example 3-1 to Example 3-4, various types such as long false eyelashes and short false eyelashes can be flexibly selected and configured with different magnetic blocks.

### Example 3-5

This example further provides a makeup removal method for the false-eyelash magnetic wearing system, including:
1. gently removing the false eyelashes from the natural eyelashes;
2. soaking a cotton pad with eye and lip makeup remover and applying it to the eye area for about 10 seconds; and
3. gently wiping the natural eyelashes, allowing the magnetic film layer to dissolve and fall off.

As an alternative, a magnetic makeup removal wand (with a built-in magnet) can be used to approach the magnetic block of the false eyelashes to assist in removing the false eyelashes, avoiding pulling.

### Application Test

### Application Example 1

150 female subjects (aged 25-40) with experience in wearing false eyelashes were recruited and randomly divided into 6 groups to be tested for wearing comfort, ease of operation, natural look, adhesion strength, and overall satisfaction of the system of the present disclosure (Examples 3-1, 3-2, 3-3, and 3-4), commercially available sandwich-style magnetic false eyelashes (Control Group 1), and commercially available eyeliner-adsorption magnetic false eyelashes (Control Group 2). Each group had a sample size of 25, and the average values were taken and rounded to one decimal place. The test results are shown in Table 1.

TABLE 1
Comparison of user experience scores for each test group

| | | | | | Control | Control |
|---|---|---|---|---|---|---|
| | | | | | Group 1 | Group 2 |
| Evaluation | Experimental | Experimental | Experimental | Experimental | (Sandwich | (Eyeliner |
| Dimension | Group (3-1) | Group (3-2) | Group (3-3) | Group (3-4) | type) | type) |
| Wearing | 9.2 | 9.0 | 9.3 | 8.8 | 3.5 | 6.2 |
| comfort | | | | | | |
| Ease of | 9.1 | 8.9 | 8.5 | 8.2 | 4.0 | 7.0 |
| operation | | | | | | |
| Natural | 9.3 | 9.1 | 9.4 | 9.0 | 5.5 | 6.5 |
| look | | | | | | |
| Adhesion | 9.0 | 9.2 | 8.8 | 9.5 | 7.0 | 4.0 |
| strength | | | | | | |
| Overall | 9.2 | 9.1 | 9.0 | 9.2 | 4.5 | 6.0 |
| Satisfaction | | | | | | |

Note: Wearing comfort: 1=extremely uncomfortable, 10=extremely comfortable; Ease of operation: 1=very complex, 10=very simple; Natural look: 1=obviously fake, 10=very natural; Adhesion strength: 1=extremely easy to fall off, 10=completely firm; Overall satisfaction: 1=very dissatisfied, 10=very satisfied.

The data in Table 1 show that the scores for the experimental group were significantly better than those of the control groups, with the advantages in comfort and naturalness being the most significant.

### Application Example 2

The eyelash magnetization fluids prepared in Examples 1-1 to 1-4 were taken to be applied evenly to simulated eyelash molds (having the same area and thickness), and after the eyelash magnetization fluids were dried into films, a Gauss meter (the probe was 0.5 mm away from the film surface) to measure the magnetic field intensity in the central area. 5 different positions were measured for each group and the average value±standard deviation was calculated. The results are shown in Table 2.

TABLE 2
Comparison of magnetic field intensity of magnetic
film layers with different formulations

| Sample | Magnetic field intensity (mT) |
|---|---|
| Example 1-1 | 8.5 ± 0.6 |
| Example 1-2 | 12.3 ± 0.7 |
| Example 1-3 | 18.7 ± 0.9 |
| Example 1-4 | 22.4 ± 1.0 |

The results in Table 2 show that the magnetic response intensities of Example 1-3 (Fe₃O₄@SiO₂ core-shell structure) and Example 1-4 (a mixture of hard-soft magnetic materials) are significantly higher than that of the single iron oxide black formulation, with increases of 120% and 163%, respectively.

### Application Example 3

The eyelash magnetization fluid prepared in Example 1-3 was placed in 45° C. high-temperature, −10° C. low-temperature, and room temperature (25° C.) environments for 3 months to observe changes in product appearance, viscosity, pH value, and magnetic response intensity. Appearance: visual inspection for abnormalities such as delamination, precipitation, discoloration, or sedimentation. Viscosity: measured using a Brookfield DV2T viscometer, No. 3 spindle, 20 rpm, at 25° C. pH value: measured directly using a pH meter (25° C.). Magnetic response intensity retention rate: the magnetic field intensity of the dried film layer was measured using the same gaussmeter as in Application Example 2, calculated as (intensity at a certain time point/initial intensity)×100%.

Three parallel samples were set for each group, and the results are shown in Table 3:

TABLE 3
Stability test results of eyelash magnetization fluids (3 months)

| | | | | | Magnetic |
|---|---|---|---|---|---|
| | | | | | response |
| | | | | | intensity |
| Test | Time | Appearance | Viscosity | pH | retention |
| conditions | point | (delamination/precipitation/discoloration) | (mPa · s) | value | rate (%) |
| 45° C. | Initial | Uniform black liquid, no abnormalities | 1850 ± 50 | 6.8 | 100% |
| | 1 month | No delamination, no precipitation, no | 1840 ± 60 | 6.7 | 98.5% |
| | | discoloration | | | |
| | 2 months | No delamination, no precipitation, no | 1820 ± 70 | 6.7 | 97.2% |
| | | discoloration | | | |
| | 3 months | No delamination, no sedimentation, no | 1800 ± 80 | 6.6 | 96.8% |
| | | discoloration | | | |
| −10° C. | Initial | Uniform black liquid, no abnormalities | 1850 ± 50 | 6.8 | 100% |
| | 1 month | No delamination, no sedimentation, no | 1860 ± 50 | 6.8 | 99.2% |
| | | discoloration | | | |
| | 2 months | No delamination, no sedimentation, no | 1870 ± 60 | 6.8 | 98.5% |
| | | discoloration | | | |
| | 3 months | No delamination, no sedimentation, no | 1860 ± 70 | 6.7 | 97.9% |
| | | discoloration | | | |
| Room | Initial | Uniform black liquid, no abnormalities | 1850 ± 50 | 6.8 | 100% |
| temperature | 1 month | No delamination, no sedimentation, no | 1845 ± 50 | 6.8 | 99.5% |
| | | discoloration | | | |
| | 2 months | No delamination, no sedimentation, no | 1840 ± 60 | 6.8 | 99.0% |
| | | discoloration | | | |
| | 3 months | No delamination, no sedimentation, no | 1835 ± 60 | 6.7 | 98.6% |
| | | discoloration | | | |

The results in Table 3 show that after 3 months at a high temperature of 45° C., the viscosity decreased slightly (about 2.7%), the pH decreased by 0.2, and the magnetic response intensity retention rate remained at 96.8%, with no delamination, precipitation, or discoloration, indicating that the product has good heat stability. Under low-temperature conditions of −10° C., the changes in all indicators after 3 months were minimal, with a magnetic response intensity retention rate of 97.9%, and no freezing damage or precipitation, indicating excellent low-temperature stability. Under room temperature conditions, all indicators remained basically stable after 3 months, with a magnetic response intensity retention rate of 98.6%, meeting conventional shelf-life requirements. In summary, the product showed no obvious delamination, precipitation, or discoloration under any of the conditions, the changes in viscosity and pH were within acceptable ranges, and the magnetic response intensity retention rate was >95%. The eyelash magnetization fluid of the present disclosure has excellent physical and chemical stability over a wide temperature range (−10° C. to 45° C.), making it suitable for commercial production, storage, and transportation.

### Application Example 4

The following safety tests were conducted on the eyelash magnetization fluid of the present disclosure (using the products prepared in Examples 1-3 as test samples) to verify its safety for use as an eye cosmetic.

### 4.1 Skin Irritation Test (Patch Test)

Test Objective: to evaluate the skin irritation of the eyelash magnetization fluid on human skin.

Test Basis: refer to the “Skin Irritation/Corrosion Test” method in the “Safety and Technical Standards for Cosmetics” (2015 Edition).

Test Samples: the eyelash magnetization fluid prepared in Examples 1-3, used as the stock solution.

Subjects: 30 healthy adult volunteers were recruited, with an equal number of males and females, aged 20-50, with no history of skin diseases, and who had not used antihistamines or glucocorticoids in the past week. All subjects signed an informed consent form.

### Test Method

Approximately 0.02 mL to 0.025 mL of the test sample was added to the filter paper of a patch tester (Finn Chamber); the patch tester was applied to the normal skin on both sides of the spine on the subjects' backs using hypoallergenic tape; a negative control (saline) and a blank control (patch tester without sample) were set for each subject simultaneously; the application time was 24 hours; skin reactions were observed 30 minutes, 24 hours, and 48 hours after removing the patch tester. Scoring criteria: refer to the skin reaction grading standards in the “Safety and Technical Standards for Cosmetics,” as shown in Table 4:

TABLE 4
Skin Reaction Grading Standards

| Reaction grade | Skin reaction |
|---|---|
| 0 | No reaction |
| 1 | Slight erythema |
| 2 | Erythema |
| 3 | Erythema + edema |
| 4 | Erythema + edema + papules |
| 5 | Severe erythema + edema + blisters |

The test results are shown in Table 5:

TABLE 5
Patch test results

| | Negative | |
|---|---|---|
| Observation time point | control | Test sample |
| 30 minutes after removal | Grade 0 | Grade 0 |
| 24 hours after removal | Grade 0 | Grade 0 |
| 48 hours after removal | Grade 0 | Grade 0 |

Conclusion: all subjects did not show irritation reactions such as erythema, edema, and papules at each observation time point, and no mechanical irritation reactions caused by particles were found, indicating that the eyelash magnetization fluid of the present disclosure is non-irritating to human skin.

### 4.2 Ophthalmic Safety Assessment (Ocular Irritation Test)

Test purpose: to evaluate the irritation of the eyelash magnetization fluid to the eyes and ensure that the product will not cause irreversible eye damage when it may come into contact with the eyes.

Test basis: refer to the “Acute ocular irritation/corrosion test” method in the “Cosmetics Safety Technical Specification” (2015 edition).

Test sample: the eyelash magnetization fluid prepared in Examples 1-3, used as the stock solution.

Experimental animals: six New Zealand white rabbits were selected, with a body weight of 2.0-2.5 kg, half male and half female. Both eyes were examined 24 hours before the experiment to confirm the absence of eye irritation symptoms. The experimental animals were housed in a standard animal room and had free access to food and water.

Test method: the eyelid of one side of the animal was gently pulled open, and 0.1 mL of the test sample was dropped into the conjunctival sac; the upper and lower eyelids were gently closed for 1 second to prevent the sample from overflowing; the other eye was left untreated as a self-control; the eyes were not washed until 24 hours after dropping the sample; the eye reactions were observed at 1 hour, 24 hours, 48 hours, 72 hours, the 4th day, and the 7th day after dropping the eye drops respectively.

Observation indicators: refer to the Draize scoring standard to score the following indicators: (1) Cornea: degree and area of turbidity; (2) Iris: degree of hyperemia and swelling; (3) Conjunctiva: degree of hyperemia, edema, and secretion status;

Scoring standard: Total score=Cornea score×5+Iris score×2+Conjunctiva score, with a maximum total score of 110 points.

Irritation degree classification: 0-3 points: non-irritating; 4-8 points: slightly irritating; 9-12 points: irritating; 13-16 points: moderately irritating; above 17 points: strongly irritating.

The test results are shown in Table 6:

TABLE 6
Results of the ocular irritation test

| Observation | | | | Total | Irritation |
|---|---|---|---|---|---|
| time | Cornea | Iris | Conjunctiva | score | level |
| 1 hour | 0 | 0 | 2 | 2 | Non-irritating |
| 24 hours | 0 | 0 | 1 | 1 | Non-irritating |
| 48 hours | 0 | 0 | 0 | 0 | Non-irritating |
| 72 hours | 0 | 0 | 0 | 0 | Non-irritating |
| Day 4 | 0 | 0 | 0 | 0 | Non-irritating |
| Day 7 | 0 | 0 | 0 | 0 | Non-irritating |

Note: a conjunctival score of 1-2 indicates mild to moderate congestion, which is a reversible reaction that completely subsides within 7 days.

Conclusion: during the 7-day observation period, no corneal or iris damage was observed in the eyes of any experimental animals; only mild conjunctival congestion occurred, which completely subsided within 48 hours. According to the Draize scoring criteria, the eye irritation classification of the eyelash magnetization fluid of the present disclosure is non-irritating.

### 4.3 Sensitization Test (Repeated Contact Sensitization Test)

Test Purpose: to evaluate whether the eyelash magnetization fluid causes skin sensitization after repeated skin contact.

Test Basis: refer to the “Skin Allergy Test” (Local Closed Patch Test) in the “Safety and Technical Standards for Cosmetics” (2015 Edition).

Test Samples: the eyelash magnetization fluid prepared in Examples 1-3, used as the stock solution.

Experimental Animals: 30 healthy white guinea pigs were selected, weighing 250-300 g, with an equal number of males and females. They were randomly divided into 3 groups: experimental group: 20 animals (applied with the test sample); positive control group: 5 animals (applied with a sensitizer 2,4-dinitrochlorobenzene); negative control group: 5 animals (applied with physiological saline). 24 hours before the experiment, the left side of the back of all animals was shaved (with an area of approximately 3 cm×3 cm).

### Test Method

1) Induction contact phase: approximately 0.1 mL of the test sample was applied to the shaved area on the left side of an animal in the experimental group; then the shaved area was covered with cellophane, occluded and secured with non-irritating tape; the positive control group was applied with 0.1 mL of 0.5% 2,4-dinitrochlorobenzene solution; the negative control group was applied with 0.1 mL of physiological saline; after 6 hours of occlusion, the covering was removed and the application site was washed with warm water; once daily for 3 consecutive days, with 6 hours of occlusion each time, for a total of 3 inductions; after the induction contact phase, the application was stopped for 2 weeks (rest period).

2) Eliciting exposure phase: on the 14th day after the end of the induction contact phase, an eliciting exposure was performed on the shaved area on the right sides of all animals (which had not been in contact with the sample); the experimental group was applied with 0.1 mL of the test sample; the positive control group was applied with 0.1 mL of 0.1% 2,4-dinitrochlorobenzene solution; the negative control group was applied with 0.1 mL of physiological saline; after 6 hours of occlusion, the covering was removed and the application site was washed with warm water; skin reactions were observed at 24 hours and 48 hours after the eliciting exposure.

Scoring Criteria: scoring was conducted according to the intensity of the skin reaction, as shown in Table 7 below:

TABLE 7
Scoring Criteria

| Sensitization reaction | Score |
|---|---|
| No reaction | 0 |
| Scattered or patchy erythema | 1 |
| Mild edema (edges identifiable) | 2 |
| Distinct erythema, edema (clear edges) | 3 |
| Severe erythema, edema (>1 mm) | 4 |

$$Sensitization rate calculation : sensitization rate = ( number of sensitized animals / total number of animals in the group ) \times 100 \% .$$

Sensitization intensity grading: Sensitization rate 0%-8%: weak sensitization; sensitization rate 9%-28%: mild sensitization; sensitization rate 29%-64%: moderate sensitization; sensitization rate 65%-80%: strong sensitization; sensitization rate 81%-100%: extreme sensitization.

The test results are shown in Table 8:

TABLE 8
Sensitization test results

| | | Number of | Number of | |
|---|---|---|---|---|
| | | sensitized | sensitized | |
| | | animals 24 | animals 48 | |
| | Number of | hours after | hours after | Sensitization |
| Group | animals | eliciting | eliciting | rate |
| Experimental | 20 | 0 | 0 | 0% |
| group | | | | |
| Positive | 5 | 5 | 5 | 100% |
| control group | | | | |
| Negative | 5 | 0 | 0 | 0% |
| control group | | | | |

Conclusion: after 24 and 48 hours of observation following the eliciting contact, no erythema, edema, or other allergic reactions were observed on the skin of any animals in the experimental group. The positive control group showed significant sensitization, while the negative control group showed no reaction. The sensitization rate of the eyelash magnetization fluid of the present disclosure is 0%, and the sensitization intensity is classified as weak sensitization (non-sensitizing).

The above test results indicate that the eyelash magnetization fluid of the present disclosure complies with the relevant requirements of the “Safety and Technical Standards for Cosmetics” regarding skin irritation, eye irritation, and skin sensitization, and possesses good safety for use as an eye cosmetic.

### Serialized Product Description:

The present disclosure can also be developed into the following series of products, specifically as follows:

(1) False Eyelashes with Different Magnetic Intensities

Strong magnetic version: using N52 grade NdFEB magnets with 5 magnetic blocks, suitable for users with thick eyelashes;

Lightweight version: using N35 grade NdFeB magnets with 2 magnetic blocks, suitable for sparse eyelashes or first-time users.

### (2) Eyelash Magnetization Fluid in Different Colors

Brown magnetic fluid: using iron oxide brown as the magnetic pigment, suitable for users with light hair color or those pursuing a natural makeup look;

Transparent magnetic fluid: using transparently coated magnetic particles (such as Fe₃O₄@SiO₂), which appear transparent after drying and can be used in combination with mascara of any color;

Colored magnetic fluid: adding cosmetic-grade colorants to the magnetic pigment to provide personalized options.

### (3) Eco-Friendly and Recyclable Design

Bio-based base strip: using polylactic acid (PLA)-based thermoplastic elastomer, which is biodegradable;

Replaceable magnetic blocks: the false eyelash base strip is equipped with magnetic block slots, allowing users to replace magnetic blocks individually to extend the service life of the false eyelashes and reduce waste.

The above descriptions are merely preferred embodiments of the present disclosure and are not intended to limit the present disclosure. For those skilled in the art, various modifications and changes can be made to the present disclosure. Any modifications, equivalent substitutions, improvements, etc., made within the spirit and principles of the present disclosure shall be included within the protection scope of the present disclosure.

What is claimed is:

1. A false-eyelash magnetic wearing system, comprising:
(a) an eyelash magnetization fluid, the eyelash magnetization fluid being prepared from the following raw materials: an acrylates copolymer, a magnetic pigment, and water, wherein a mass content of the acrylates copolymer in the raw materials is 10%-30%, a mass content of the magnetic pigment in the raw materials is 50%-80%, with the balance being water;
(b) magnetic block-equipped false eyelashes, comprising:
a non-magnetic flexible base strip;
false eyelash fibers fixed to the flexible base strip; and
a plurality of permanent magnet blocks fixed to the flexible base strip, the plurality of permanent magnet blocks being arranged along a length direction of the flexible base strip, and the permanent magnet blocks being configured to generate a magnetic attraction force with natural eyelashes treated by the eyelash magnetization fluid.

2. The false-eyelash magnetic wearing system according to claim 1, wherein in the eyelash magnetization fluid, the mass content of the acrylates copolymer in the raw materials is 20%-30%; and/or, the mass content of the magnetic pigment in the raw materials is 65%-75%.

3. The false-eyelash magnetic wearing system according to claim 1, wherein the magnetic pigment is selected from a group consisting of: black iron oxide, fatty acid surface-treated black iron oxide, nano-scale ferroferric oxide, composite magnetic material, or a mixture of a hard magnetic material and a soft magnetic material; wherein the composite magnetic material is a Fe₃O₄@SiO₂ core-shell structure material; wherein the hard magnetic material is at least one of barium ferrite or strontium ferrite; and the soft magnetic material is cobalt-nickel ferrite.

4. The false-eyelash magnetic wearing system according to claim 3, wherein the nano-scale ferroferric oxide has a particle size from 20 to 100 nm; or the fatty acid is selected from at least one of lauric acid or oleic acid; or in the mixture of a hard magnetic material and a soft magnetic material, a mass percentage of the hard magnetic material to the soft magnetic material is 60%-80%: 20%-40%.

5. The false-eyelash magnetic wearing system according to claim 1, wherein the raw materials of the eyelash magnetization fluid further comprise a humectant, a surfactant, and a pH regulator; wherein the humectant is selected from at least one of propylene glycol, butylene glycol, glycerin, dipropylene glycol, PEG400, sorbitol, sodium hyaluronate, sodium PCA, trehalose, or methyl gluceth-20, with a mass content of 2%-6% in the raw materials; the surfactant is selected from at least one of polysorbate-80, poloxamer 188, poloxamer 407, lecithin, diethylhexyl phosphate, dibutyl phosphate, or lauric acid, with a mass content of 1%-3% in the raw materials; and the pH regulator is selected from arginine, with a mass content of 0.5%-2% in the raw materials.

6. The false-eyelash magnetic wearing system according to claim 5, wherein the humectant is selected from: a combination of butylene glycol with a mass content of 1%-2% and glycerin with a mass content of 1%-2% in the raw materials, or a combination of 1%-2% methyl gluceth-20, 0.1%-0.2% sodium hyaluronate, and 2%-3% butylene glycol; and/or, the surfactant is selected from a combination of poloxamer 188 with a mass content of 1%-2% and lecithin with a mass content of 0.5%-1% in the raw materials.

7. The false-eyelash magnetic wearing system according to claim 1, further comprising a preservative, wherein the preservative is selected from at least one of ethylhexylglycerin and caprylyl glycol, with a mass content of 0.1%-1% in the raw materials.

8. The false-eyelash magnetic wearing system according to claim 1, wherein the raw materials of the eyelash magnetization fluid comprise, by mass percentage: 22% to 25% of an acrylates copolymer, 65% to 68% of a magnetic pigment, 2%-6% of a humectant, 2%-3% of a surfactant, 0.8%-1.5% of a pH regulator, 0.1%-0.5% of a preservative, with the balance being water.

9. The false-eyelash magnetic wearing system according to claim 8, wherein a method for preparing the eyelash magnetization fluid comprises the following steps:
Step 1: mixing the acrylates copolymer with water, then adding the magnetic pigment and mixing;
Step 2: mixing a mixture from step 1 at a speed of 800-8000 rpm for 5-15 min;
Step 3: further adding the humectant, the surfactant, and the pH regulator to the mixture and mixing, and finally adding the preservative and mixing;
Step 4: allowing the mixture to stand, and filtering out solids to obtain the eyelash magnetization fluid.

10. The false-eyelash magnetic wearing system according to claim 9, wherein in the step 2, the mixture from step 1 is mixed using a high-speed shear emulsification process at a speed of 3000-6000 rpm for 5-8 min; and/or,
in the step 4, a 200-400 mesh screen is used for filtration to remove large particle agglomerates and impurities; and/or,
a vacuum defoamation treatment is added after the step 2 or the step 3.

11. The false-eyelash magnetic wearing system according to claim 1, wherein a material of the non-magnetic flexible base strip is silicone, thermoplastic elastomer, or fabric; wherein the thermoplastic elastomer is selected from at least one of thermoplastic polyurethane elastomer, thermoplastic polyolefin elastomer, thermoplastic polyester elastomer, and thermoplastic vulcanizate; and wherein the fabric is selected from one of natural fiber fabric, synthetic fiber fabric, non-woven fabric, and blended fabric.

12. The false-eyelash magnetic wearing system according to claim 1, wherein the permanent magnet blocks are arranged at intervals along a length direction of the flexible base strip; the permanent magnet blocks are wrapped or bonded within the flexible base strip, or fixed to a surface of the flexible base strip; and a number of the permanent magnet blocks is 2 to 5.

13. The false-eyelash magnetic wearing system according to claim 12, wherein an anti-corrosion coating, preferably a nickel-plated or gold-plated layer, is provided on a surface of the permanent magnet block; or the permanent magnet block is coated with a medical-grade silicone layer.

14. The false-eyelash magnetic wearing system according to claim 12, wherein the permanent magnet block has a magnetic micro-array structure, comprising a plurality of magnetic microcolumns or microgrooves; and magnetic pole directions of the plurality of permanent magnet blocks are arranged alternately.

15. A method for wearing false eyelashes using the false-eyelash magnetic wearing system according to claim 1, comprising the following steps:
applying the eyelash magnetization fluid onto a user's natural eyelashes;
bringing the permanent magnet blocks of the magnetic block-equipped false eyelash close to the natural eyelashes coated with the eyelash magnetization fluid, and adsorbing and fixing the magnetic block-equipped false eyelashes onto the natural eyelashes through magnetic force.

16. The method according to claim 15, wherein the method further comprises a step of adsorbing and fixing the magnetic block-equipped false eyelashes onto an upper or lower side of the natural eyelashes according to makeup requirements.

17. The method according to claim 15, wherein the method further comprises segmented wearing: selecting false eyelashes with different numbers of magnetic blocks for separate wearing according to different regions of an inner corner, a middle portion, and an outer corner of an eye; or combined wearing: wearing the magnetic block-equipped false eyelashes onto both the upper and lower sides of the natural eyelashes simultaneously.

18. The method according to claim 15, wherein the method further comprises a makeup removal step: using an eye and lip makeup remover to wipe off the eyelash magnetization fluid on the natural eyelashes, or using a magnetic makeup removal wand to assist in removing the false eyelashes.

19. A method for preparing the eyelash magnetization fluid used in the false-eyelash magnetic wearing system according to claim 1, comprising:
step (1), mixing the acrylates copolymer with water, and then adding the magnetic pigment and mixing;
step (2), mixing a mixture obtained in step (1) at a rotation speed of 800-8000 rpm for 5-15 min;
Step (3), afterwards leaving a mixture obtained in step (2) to stand, and obtaining the eyelash magnetization fluid after filtering out solids.

20. The method according to claim 19, wherein the eyelash magnetization fluid further comprises a humectant, a surfactant, a pH regulator, and a preservative; and the preparation method further comprises: after the step (2), adding and mixing the humectant, the surfactant, and the pH regulator, then adding and mixing the preservative; and then performing the step (3); and
a vacuum defoamation treatment is added after the step (2) or the step (3).
